(19) United States
(12) Patent Application Publication
(10) Pub. No.: US 2023/0355895 A1
(43) Pub. Date: Nov. 9, 2023

(54) CONTROLLED DISPENSE SYRINGE

(71) Applicant: AMGEN INC., Thousand Oaks, CA (US)

(72) Inventors: Samin Akbari, Cambridge, MA (US); Paul Daniel Faucher, Thousand Oaks, CA (US)

(21) Appl. No.: 17/911,510

(22) PCT Filed: Feb. 8, 2021
(86) PCT No.: PCT/US21/17003

§ 371 Date: Sep. 14, 2022

Related U.S. Application Data
(60) Provisional application No. 62/990,819, filed on Mar. 17, 2020.

(51) Int. Cl. A61M 5/36; A61M 5/315; A61M 5/31

(52) U.S. Cl. CPC A61M 5/36; A61M 5/31505; A61M 5/3146; A61M 2005/31508

(57) ABSTRACT

A syringe is described with a plunger assembly that is adapted to have a dispensing stroke sized to dispense a fluid therapeutic product from the syringe without dispensing any air or headspace. The syringe includes a plunger rod having a stop feature that stops a dispensing stroke of the plunger rod at a distance corresponding to a level of air or headspace within the syringe avoiding additional air dispensing steps from a healthcare provider.

### CROSS-REFERENCE TO RELATED APPLICATION

Priority is claimed to U.S. Provisional Patent Application No. 62/990,819, filed Mar. 17, 2020, the entire contents of which are hereby incorporated herein by reference.

### FIELD OF THE DISCLOSURE

The present disclosure generally relates to syringes and, more particularly, to prefilled syringes.

### BACKGROUND

For some drug dispensing devices, a healthcare provider is required to transfer a therapeutic product from a prefilled syringe to a cartridge or device reservoir that is configured for the drug dispensing device. Typically, a prefilled syringe is filled with a predetermined amount of the therapeutic product along with a predetermined amount of air. To transfer the therapeutic product to the cartridge or device reservoir, a healthcare provider can orient the prefilled syringe so that the air is adjacent to the needle and depress a plunger of the prefilled syringe to dispense the air. This air dispensing operation may inadvertently dispense some of the therapeutic product, which can waste an expensive product and may cause the amount of therapeutic product in the prefilled syringe to fall below a prescribed amount for the patient. On the other hand, if the air is not fully dispensed from the prefilled syringe, some air may be transferred to the cartridge or device reservoir. Air within a fluid flow path of the drug dispensing device can negatively impact the operation of the device, such as by causing uncontrolled forward or backward flow due to barometric pressure changes.

### SUMMARY

In accordance with a first aspect, a syringe is disclosed herein that includes a barrel having an interior, a dispensing opening at a distal end, and an open proximal end; a stopper disposed within the interior of the barrel; a plunger rod having a first end configured to be at least selectively operably coupled with the stopper and a second end extending through the open proximal end of the barrel, where the second end includes an outwardly extending flange; and a stop assembly including a plunger stop coupled to the plunger rod at a location between the flange and the open proximal end of the barrel and a removable portion coupled to the plunger rod or barrel. The plunger stop is configured and dimensioned to engage a stop surface adjacent to the open proximal end of the barrel to selectively limit displacement of the plunger and stopper along the interior of the barrel

According to one form, the plunger stop and the removable portion of the stop assembly can include first and second bodies that are pivotable with respect to one another to capture the plunger rod therebetween. In this form, the plunger stop and the removable portion of the stop assembly can be a clip that includes a hinge pivotably coupling first sides of the first and second bodies and a securing mechanism releasably connecting second sides of the first and second bodies. Alternatively, in this form, the plunger stop and the removable portion of the stop assembly can be a clamp that includes a biasing mechanism and the first and second bodies can include jaws at first ends thereof and handles at second ends thereof, where the biasing mechanism biases the jaws of the first and second bodies together to capture the plunger rod therebetween.

According to one form, the plunger stop and the removable portion of the stop assembly can include a body defining a cavity with an opening through an end wall thereof, where the cavity is configured to receive the plunger rod therein to restrict longitudinal movement of the plunger rod with respect to the body, and a securing mechanism coupled to the body to hold the plunger rod within the cavity. In this form, the securing mechanism can be an adhesive member extending over cavity. Alternatively, in this form, the body can include first and second clamshell components having inner faces configured to be brought together to define the cavity therebetween; and the securing mechanism can hold the first and second clamshell components in a closed configuration with the plunger rod trapped within the cavity.

According to one form, the proximal end of the barrel can include an outwardly projecting flange and the plunger stop can be a stop flange that extends outwardly from the plunger rod adjacent to the first end thereof and has a perimeter sized to fit within the interior of the barrel. The removable portion of the stop assembly can be a blocking member that is configured to removably engage the flange of the barrel, such that the blocking member has a portion disposed within a travel path of the stop flange to restrict longitudinal movement of the plunger rod. In this form, the blocking member can be a clip having first and second walls spaced from one another and connected by an end wall, where the clip is configured to engage the flange of the barrel between the first and second walls. Alternatively, in this form, the blocking member can be a tab having a distal end with a configuration complementary to a portion of a horizontal cross-section of the plunger rod, and the flange of the barrel can include a channel that is sized to receive the tab therein, where the channel is configured to guide the tab into engagement with the plunger rod.

According to other forms, the plunger stop and the removable portion of the stop assembly can be a snap-fit member that includes first and second walls spaced from one another and connected by an end wall, where the first and second walls define channels on interior faces thereof and are configured to be flexed apart to dispose the plunger rod therebetween and returned towards an unflexed position to retain portions of the plunger rod within the channels; or can be a tab coupled to the plunger rod along a longitudinal length thereof by a breakaway feature.

According to any of the above forms, the stop assembly can include a wall portion having a face with information displayed thereon regarding the operation of the stop.

In accordance with a second aspect, a method of dispensing headspace from a syringe containing a liquid therapeutic product is disclosed that includes providing a syringe comprising: a barrel having an interior, a dispensing opening at a distal end, and an open proximal end; a stopper disposed within the interior of the barrel; a plunger rod having a first end configured to be at least selectively operably coupled with the stopper and a second end extending through the open proximal end of the barrel, wherein the second end includes an outwardly extending flange; a fluid therapeutic product and a headspace disposed in the interior of the barrel between the stopper and the dispensing opening; and a stop assembly including a plunger stop coupled to the plunger rod and a removable portion coupled to the plunger rod or barrel. The method further includes driving the plunger rod toward the open proximal end of the barrel of the syringe to drive the stopper along the interior of the barrel to dispense the headspace through the dispensing opening, abutting the plunger stop with a stop surface adjacent to the open proximal end of the barrel, thereby stopping movement of the stopper and preventing the fluid therapeutic product from being expelled, and removing the removable portion of the stop assembly from the plunger rod or barrel.

According to some forms, the method can include one or more of the following aspects: the plunger stop and the removable portion of the stop assembly can include first and second bodies having the plunger rod captured therebetween and removing the removable portion of the stop assembly from the plunger rod or barrel can include pivoting first and second bodies of the stop with respect to one another; removing the removable portion of the stop assembly from the plunger rod or barrel can include breaking a tab off of the plunger rod; the plunger stop and the removable portion of the stop assembly can include a body defining a cavity with an opening through an end wall thereof, where the plunger rod is received within the cavity, and a securing mechanism coupled to the body to hold the plunger rod within the cavity, and removing the removable portion of the stop assembly from the plunger rod or barrel can include manipulating the body and securing mechanism to free the plunger rod from the cavity; removing the removable portion of the stop assembly from the plunger rod or barrel can include flexing first and second walls of the stop assembly away from one another to release the plunger rod from being captured therebetween; or the proximal end of the barrel can include an outwardly projecting flange, the plunger stop can be a stop flange extending outwardly from the plunger rod, where the stop flange has a perimeter sized to fit within the interior of the barrel, and removing the removable portion of the stop assembly from the plunger rod or barrel can include uncoupling a blocking member having a portion disposed within a travel path of the stop flange to restrict longitudinal movement of the plunger rod from the flange of the barrel.

In accordance with a third aspect, a syringe is disclosed that includes a barrel having an interior, a dispensing opening at a distal end, and an open proximal end having an end surface; a stopper disposed within the interior of the barrel; a plunger rod having a first end configured to be at least selectively operably coupled with the stopper and a second end extending through the open proximal end of the barrel; an outwardly projecting stop adjacent the second end of the plunger rod; an engagement portion at the second end of the plunger rod spaced from the stop; and at least one wall portion that extends longitudinally between the stop and the engagement portion and laterally outward from the plunger rod, where the at least one wall portion is integrally formed with the plunger rod and has a face with information displayed thereon regarding the operation of the stop.

In some forms, the at least one wall portion can be wall portions extending laterally outward from opposing sides of the plunger rod.

In accordance with a fourth aspect, a syringe is disclosed that includes a barrel having an interior, a dispensing opening at a distal end, and an open proximal end having an end surface; a stopper disposed within the interior of the barrel; a plunger rod having a first end configured to be at least selectively operably coupled with the stopper and a second end extending through the open proximal end of the barrel; an engagement portion at the second end of the plunger rod; a flange adjacent to the second end of the plunger and spaced from the engagement portion; and a snap-fit member releasably coupled to the plunger rod. The snap-fit member includes first and second walls that are spaced from one another and connected by an end wall, where the first and second walls are configured to be flexed apart to dispose the plunger rod therebetween and returned towards an unflexed position to retain the snap-fit member on the plunger rod between the engagement portion and the flange.

In some forms, the first and second walls of the snap-fit member define channels on interior faces thereof, and the channels are configured to receive portions of the plunger rod therein with the first and second walls in the unflexed position.

### BRIEF DESCRIPTION OF THE DRAWINGS

FIG. 1 is a perspective view of a first example syringe in a full configuration with a stop assembly showing a fluid therapeutic product and headspace within a barrel according to an embodiment of the present disclosure.

FIG. 2 is a side elevational view of the syringe of FIG. 1 in the full configuration.

FIG. 3 is a side elevational view of the syringe of FIG. 1 in a dispensed configuration showing the fluid therapeutic product within the barrel.

FIG. 4 is a side elevational view of the syringe of FIG. 1 showing a removable portion of the stop assembly in an uncoupled configuration.

FIG. 5 is a perspective view of a second example syringe in a full configuration with a stop assembly showing a fluid therapeutic product and headspace within a barrel according to an embodiment of the present disclosure.

FIG. 6 is a perspective view of a third example syringe in a full configuration with a stop assembly showing a fluid therapeutic product and headspace within a barrel according to an embodiment of the present disclosure.

FIG. 7 is a perspective view of the syringe of FIG. 6 showing a removable portion of the stop assembly in an uncoupled configuration.

FIG. 8 is a perspective view of a fourth example syringe in a full configuration with a stop assembly showing a fluid therapeutic product and headspace within a barrel according to an embodiment of the present disclosure.

FIG. 9 is a perspective view of the syringe of FIG. 8 showing a removable portion of the stop assembly in an uncoupled configuration.

FIG. 10 is a perspective view of a fifth example syringe in a full configuration with a stop assembly showing a fluid therapeutic product and headspace within a barrel according to an embodiment of the present disclosure.

FIG. 11 is a perspective view of a sixth example syringe in a full configuration with a stop assembly showing a fluid therapeutic product and headspace within a barrel according to an embodiment of the present disclosure.

FIG. 12 is an exploded view of the syringe of FIG. 11 showing a removable portion of the stop assembly in an uncoupled configuration.

FIG. 13 is a perspective view of a seventh example syringe in a full configuration with a stop assembly showing a fluid therapeutic product and headspace within a barrel according to an embodiment of the present disclosure.

FIG. 14 is a detail view of the syringe of FIG. 13.

FIG. 15 is a perspective view of an eighth example syringe in a full configuration with a stop assembly showing a fluid therapeutic product and headspace within a barrel according to an embodiment of the present disclosure.

FIG. 16 is an exploded view of the syringe of FIG. 15 showing a removable portion of the stop assembly in an uncoupled configuration.

FIG. 17 is a perspective view of a ninth example syringe in a full configuration with a stop assembly showing a fluid therapeutic product and headspace within a barrel according to an embodiment of the present disclosure.

FIG. 18 is a front elevational view of the syringe of FIG. 17 in the full configuration.

FIG. 19 is a front elevational view of the syringe of FIG. 17 in a dispensed configuration with the headspace in the barrel.

FIG. 20 is a perspective view of a tenth example syringe in a dispensed configuration with a stop assembly showing headspace within a barrel according to an embodiment of the present disclosure.

FIG. 21 is an exploded, detail view of the syringe of FIG. 20 showing a removable portion of the stop assembly in an uncoupled configuration.

### DETAILED DESCRIPTION

Syringes and syringe assemblies are provided with a plunger assembly that is adapted to provide a dispensing stroke that controls a headspace within the syringe. The control of the headspace can include purging the headspace prior to dispensing a therapeutic product or dispensing a therapeutic product without dispensing any headspace. More specifically, the syringe includes a stop or stop assembly that stops a dispensing stroke of the plunger rod at a predetermined distance corresponding to a level of headspace within the syringe. The distance can be utilized to provide controlled dispensing of the headspace or can be configured such that a stroke of the plunger rod leaves the headspace within the syringe. Accordingly, a syringe having such a configuration allows a practitioner to safely address or skip the step of expelling air bubbles from the syringe.

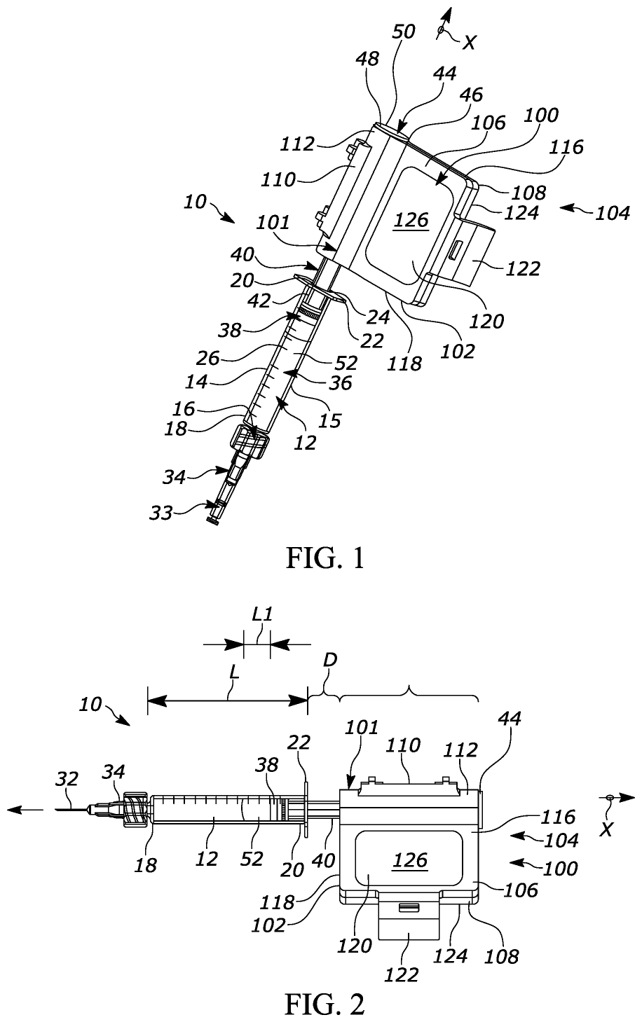

Referring now to the drawings, syringes 10 are shown extending along a longitudinal axis X. The syringes 10 are illustrated in a pre-filled state having a dose or other predetermined amount of a fluid therapeutic product 12 contained within a barrel or reservoir 14. The barrel 14 has tubular configuration oriented along the longitudinal axis X with an annular sidewall 15 extending between a dispensing opening 16 at a distal end 18 and an open proximal end 20. The proximal end 20 can include a radially projecting flange 22 that provides an upwardly facing end surface 24. If desired, the barrel 14 can include measurement markings 26 distributed along a length L thereof to provide a visual indication of the amount of product 12. The barrel 14 can be made of any suitable material, such as glass or a polymer. At the distal end 18 of the barrel 14, the syringe 10 includes a needle 32 and a needle hub 34 coupled to the barrel 14, such that the needle 32 is fluidly coupled with an interior 36 of the barrel 14. If desired, the syringe 10 can further include a rigid or non-rigid needle shield 33 (FIG. 1) that is disposed over at least a portion of a distal end of the needle 32. The syringe 10 further includes a stopper (plunger-stopper) 38 and a plunger rod 40 that is configured to at least selectively engage the stopper 38 and project outwardly through the open proximal end 20 of the barrel 14 along the longitudinal axis X. The plunger rod 40 includes an elongate body 41 that is coaxial with the barrel 14. The body 41 engages the stopper 38 at a distal end 42 and includes a thumb rest or engagement portion 44 at an opposite, proximal end 46. The plunger rod 40 can be at least selectively operably coupled with the stopper 38 by any suitable method, such as embedded therein, having mating components, abutting an end surface, selectively disposed within a recess of the stopper 38, and so forth. For example, the plunger rod 40 and stopper 38 can have a retractable or non-retractable configuration, as desired. As such, the plunger rod 40 can be coupled with the stopper 38, such that retraction of the plunger rod 40 through the barrel 14 also causes the stopper 38 to be pulled rearwardly along the barrel 14 or can be selectively coupled with the stopper 38, such that retraction of the plunger rod 40 through the barrel 14 does not cause a corresponding retraction of the stopper 38. The plunger rod 40 can As shown, the engagement portion 44 can include a flange 48 that extends outwardly from the body 41 generally perpendicular to the longitudinal axis X providing a user with a flat or contoured upwardly facing engagement surface 50. So configured, a user can grip the flange 22 of the proximal end 20 of the barrel 14 with two fingers and the engagement surface 50 with a thumb to press down and drive the stopper 38 through the barrel 14 in a dispensing operation.

As shown, the barrel 14 also contains headspace 52, e.g., air, along with the product 12. Traditionally, a healthcare provider handles the syringe 10 so that the needle 32 is oriented upwardly and depresses the plunger rod 40 to drive the stopper 38 through the barrel 14 to dispense the headspace 52 through the needle 32 or eyes the level of product 12 during dispensing to avoid expelling any headspace 52.

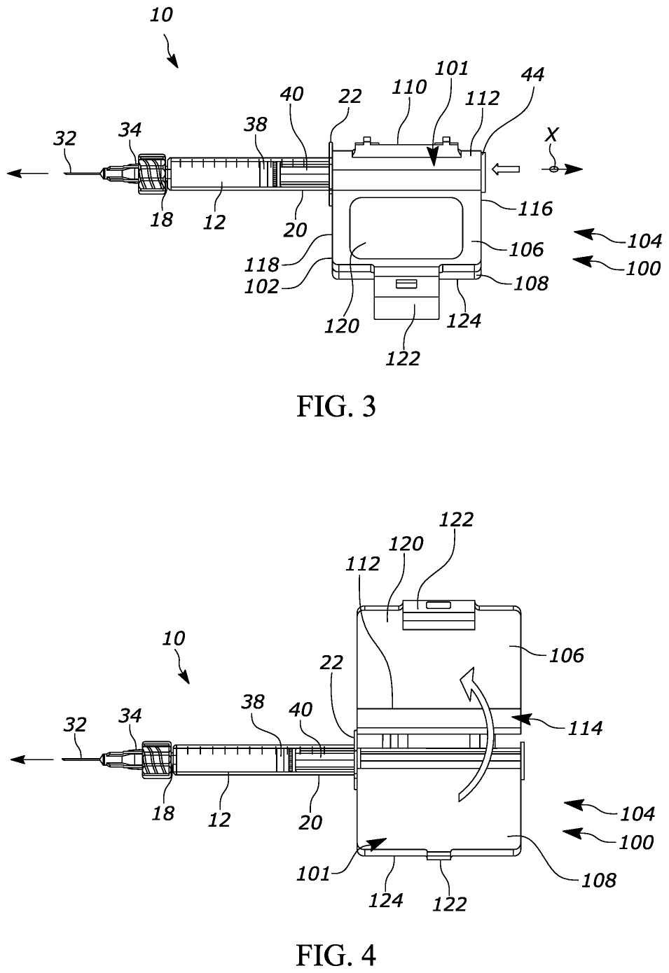

In first examples, shown in FIGS. 1-16, the syringe 10 is prepared for use with the following steps. In a first step, the practitioner tilts the syringe 10 upward to visually inspect the syringe 10 and observe the headspace 52 migrating toward the dispensing opening 16 of the barrel 14. In a second step, if applicable, the practitioner removes the needle shield 33 to expose distal end of the needle 32. In a third step, the practitioner pushes on the plunger rod 40 to expel the headspace 52.

As shown, each of the syringes 10 of the embodiments shown in FIGS. 1-16 include a stop assembly 100 that restricts a distance that the plunger rod 40 can be pushed into the barrel 14. Specifically, the stop assembly 100 only allows the plunger rod 40 to be pushed into the barrel 14 a distance that purges the headspace 52. It will be understood that purging the headspace 52 from the barrel 14 can include dispensing a small amount of the product 12 from the syringe 10 along with the headspace 52 to ensure that the headspace 52 has been fully purged. Advantageously, the stop assembly 100 prevents excess amounts of the product 12 from being dispensed during the headspace purging step. Next, in a fourth step, the practitioner removes or disables a removable portion 101 of the stop assembly 100 and, in a fifth step, administers the product 12 according to normal operating procedure.

In each of these examples, the stop assembly 100 has a plunger stop 102 that is or can be spaced from the proximal end 20 of the barrel 14 a distance D that corresponds to a length L1, and corresponding volume, of the headspace 52 within the barrel 14. It will be understood that the L1 can be sized to include a small amount, e.g., 2%-10% of a delivered mL amount, of product 12 to ensure that the headspace 52 is fully purged. With this configuration, a user can push the plunger rod 40 to drive the stopper 38 through the barrel 14 and the plunger stop 102 will abut the barrel 14 or a structure associated with the barrel 14 adjacent to the open proximal end 20 to prevent further movement of the plunger rod 40 and the stopper 38. Accordingly, the stop assembly 100 ensures that the plunger rod 40 is moved a sufficient distance to purge the headspace 52 without dispensing excess product 12. Stated another way, by configuring the plunger rod 40 and stop assembly 100 to result in a movement of the plunger rod 40 a predetermined length that corresponds to a desired portion of the barrel 14, the syringe 10 can be preconfigured to dispense a specific volume of the syringe 10 corresponding to the headspace 52 within conventional pre-filled syringes.

A first example syringe 10 is shown in FIGS. 1-4. In this form, the stop assembly 100, including the removable portion 101 and the plunger stop 102, is provided by a clip 104 having first and second bodies 106, 108 pivotably coupled together by a hinge or other pivot 110. The first and second bodies 106, 108 are pivotable with respect to one another to removably mount the clip 104 to the plunger rod 40. As shown, the bodies 106, 108 each include curved portions 112, such that when the second bodies 106, 108 are in a closed configuration, the curved portions 112 define a through bore 114 sized to allow the plunger rod 40 to extend through the clip 104. The bore 114 is sized so that the clip 104 is retained between the flange 48 of the engagement portion 44 and the barrel 14, with an upper surface 116 of the clip 104 configured to abut the flange 48 of the engagement portion 44 and a lower surface 118 of the clip 104 configured to abut the end surface 24 of the barrel proximal end 20 after initial displacement. Accordingly, in this form, the lower surface 118 of the clip 104 provides the plunger stop 102 of the stop assembly 100. With this configuration, if a user pushes on the plunger rod 40, the clip 104 restricts full movement of the plunger rod 40 to dispense the product 12. For example, the bore 114 can have at least one dimension, e.g., diameter, smaller than the flange 48 of the engagement portion 44 so that the clip 104 is retained on the plunger rod 40 by the upper surface 116 hitting the flange 48. Additionally, the clip 104 can be prevented from transferring to the barrel 14 by the flange 22 abutting the clip 104 and/or the bore 114 having a dimension, e.g. diameter, smaller than the barrel 14 so that the lower surface 118 of the clip 104 abuts the end surface 24 of the barrel 14.

As shown, the curved portions 112 of the bodies 106, 108 can be disposed on a lateral side thereof and the bodies 106, 108 can further include generally planar portions 120 that extend laterally away from the curved portions 112. With the bodies 106, 108 in the closed configuration, the planar portions 120 extend along and abut one another, such that the bore 114 is closed off on sides thereof by the hinge 110 and the planar portions 120. The clip 104 can include a latch or other securing mechanism 122, such as a snap-fit, having complementary components on lateral edges 124 of the planar portions 120. The latch 122 allows the clip 104 to be attached to and removed from the plunger rod 40. Moreover, main faces 126 of the planar portions 120 can have instructions, drug identification information, or other information thereon for easy viewing by a user. For example, the information can inform a user as to the proper operation and use of the clip 104. The information can be printed directly on the planar portions 116 and/or can be applied thereto, such as with a sticker or the like.

Accordingly, the clip 104 can be attached to the plunger rod 40 and after a practitioner inspects and turns the syringe 10 upward, the practitioner can push on the plunger rod 40 until the clip 104 restricts further movement thereof by extending between the proximal end 20 of the barrel 14 and the flange 48 of the engagement portion 44. Thereafter, the clip 104 can be removed by uncoupling the latch 122 and pivoting the bodies 106, 108 away from one another. After the clip 104 is removed, the plunger rod 40 is unrestricted for a normal administration procedure.

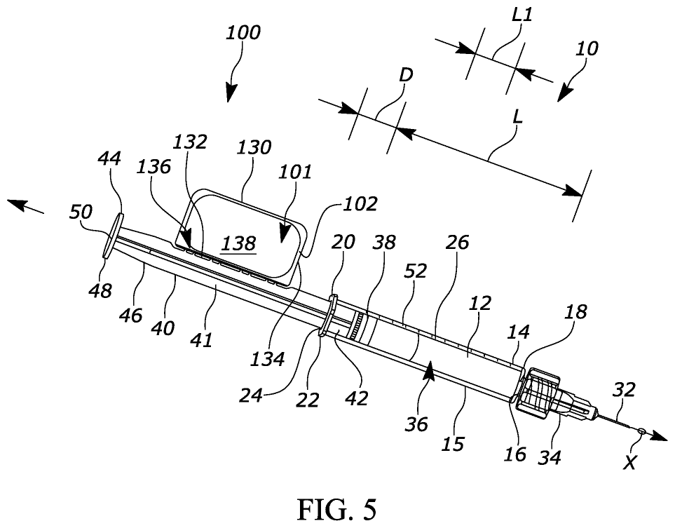

A second example syringe 10 is shown in FIG. 5. In this form, the stop assembly 100, including the removable portion 101 and the stop flange 102, is provided by a tab 130 removably and rigidly fixed to the plunger rod 40. The tab 130 extends along a longitudinal length of the plunger rod 40 and is fixed thereto along a side edge 132. As shown, a lower edge 134 of the tab 130 provides the plunger stop 102 of the stop assembly 100 to restrict movement of the plunger rod 40. For example, the tab 130 can be fixed to the plunger rod 40 so that the lower edge 134 is spaced from the proximal end 20 of the barrel 14 prior to use and is configured to abut the proximal end 20 of the barrel 14 after the plunger rod 40 has traveled the distance D with respect to the barrel 14 to purge the headspace 52. With this configuration, if a user pushes on the plunger rod 40, the tab 130 restricts full movement of the plunger rod 40 to prevent the product 12 from being dispensed.

Thereafter, the tab 130 can be broken off or removed from the plunger rod 40 to allow the plunger rod 40 to move freely. If desired, a connection 136 between the tab 130 and the plunger rod 40 can be defined by an easy-break feature, such as perforations or breaks in the material, extending between the tab 130 and the plunger rod 40. So configured, after use, a user can easily bend the tab 130 with respect to the plunger rod 40 to break the connection between the tab 130 and the plunger rod 40 to thereby leave the plunger rod 40 unrestricted for a normal administration procedure. In one example, the plunger rod 40 and tab 130 can be integrally formed as a single piece component. Moreover, main faces 138 of the tab 130 can have instructions, drug identification information, or other information thereon for easy viewing by a user. For example, the information can inform a user as to the proper operation and use of the tab 130. The information can be printed directly on one or both of the faces 138 and/or can be applied thereto, such as with a sticker or the like.

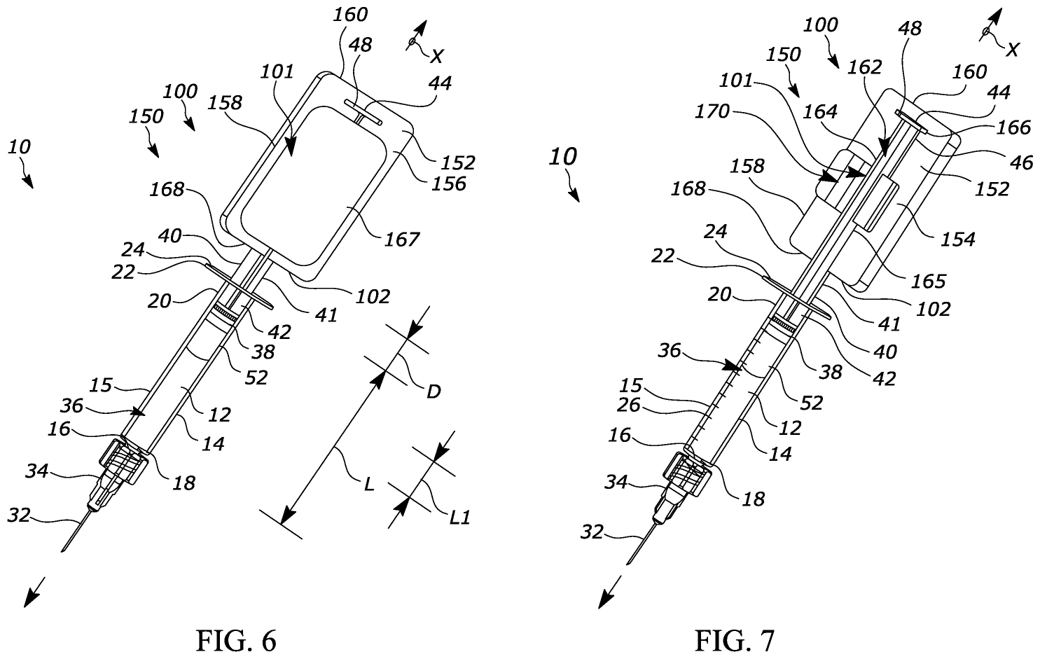

A third example syringe 150 is shown in FIGS. 6 and 7. In this form, the stop assembly 100, including the removable portion 101 and the plunger stop 102, is provided by a block 150 having a body 152 with front and rear main faces 154, 156 defined by side edges 158 and end edges 160 and a securing mechanism 170. A cavity 162 is formed in body 152 with an opening 164 to the cavity 162 extending across the front main face 154 and an end wall 168 of the block 150. As shown, the cavity 162 has a shape corresponding to the proximal end of the plunger rod 40 with a rod portion 165 sized to receive the rod 40 therein and a thumb rest portion 166 sized to receive the engagement portion 44 therein. The shape of the cavity 162 restricts longitudinal movement of the plunger rod 40 with respect to the block 150 due to the T-shape abutting the engagement portion 44. In one version, the thumb rest portion 166 of the cavity 162 can extend entirely through the block 150, which allows the body 152 to have a reduced depth. Additionally, if desired, the rod portion 165 of the cavity 162 can extend entirely through the block 150 and the block 150 can include a retaining member 167 that extends along the rear main face 156 to extend over at least a portion of the rod portion 164. The rear main face 156 or the retaining member 167 thereon can have instructions, drug identification information, or other information thereon for easy viewing by a user. For example, the information can inform a user as to the proper operation and use of the block 150. The information can be printed directly on the face 156 or retaining member 167 and/or can be applied thereto, such as with a sticker or the like.

The body 152 is sized to extend along a length of the plunger rod 40 so that the end wall 168 of the block 150 provides the plunger stop 102 of the stop assembly 100 to restrict movement of the plunger rod 40. For example, the end wall 168 can be spaced from the proximal end 20 of the barrel 14 prior to use and can be configured to abut the proximal end 20 of the barrel 14 after the plunger rod 40 has traveled the distance D with respect to the barrel 14 to purge the headspace 52. With this configuration, if a user pushes on the plunger rod 40, the block 150 prevents full movement of the plunger rod 40 to prevent the product 12 from being dispensed.

The plunger rod 40 can be retained within the cavity 162 by the securing mechanism 170. In one example, the securing mechanism 170 can be an adhesive member, such as tape or a sticker, that secures to the block 150 and extends over at least part of the opening 164 to the cavity 162, e.g., the rod portion 165. So configured, after the headspace 52 has been purged, a user can at least partially remove the adhesive member 170 and take the plunger rod 40 out of the cavity 162. In another example, the securing mechanism 170 can be an elastic member that extends around the block 150.

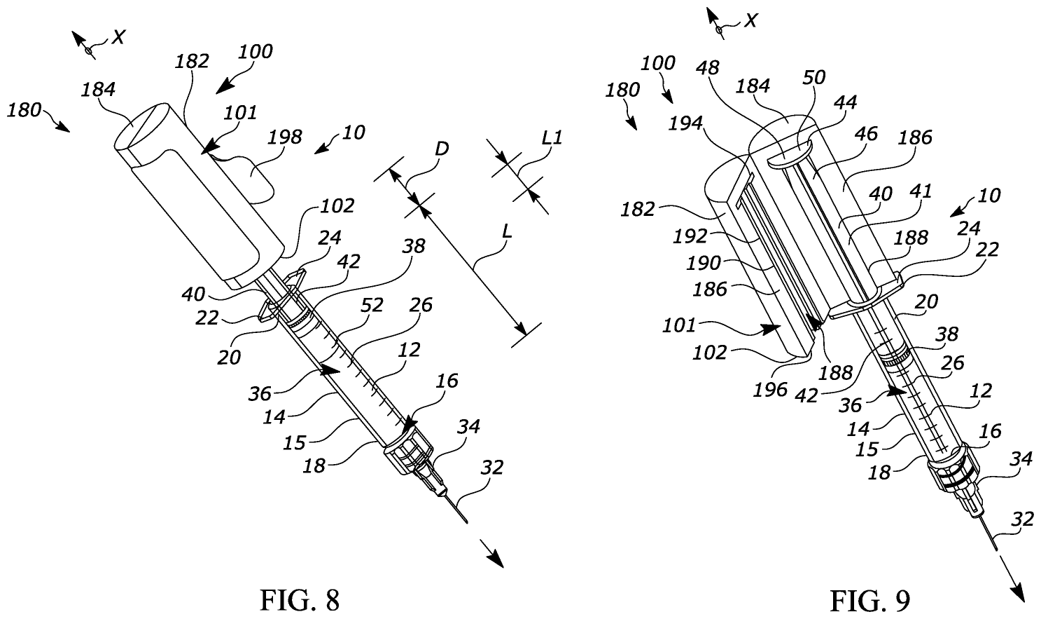

A fourth example syringe 10 is shown in FIGS. 8 and 9. In this form, the stop assembly 100, including the removable portion 101 and the plunger stop 102, is provided by a clamshell member 180 and a securing mechanism 198. The clamshell member 180 has first and second bodies 182, 184 with interior faces 186 configured to extend along and abut one another when the member 180 is in a closed configuration. Each of the bodies 182, 184 defines a cavity 188 having an opening 190 extending across the interior face 186 and an end wall 196 of body 182, 184. As shown, the cavities 188 have a shape corresponding to half of the proximal end of the plunger rod 40 with a rod portion 192 sized to receive the rod 40 therein and a thumb rest portion 194 sized to receive the engagement portion 44 therein. The shape of the cavities 188 restricts longitudinal movement of the plunger rod 40 with respect to the member 180 due to the T-shape abutting the engagement portion 44. As such, the bodies 182, 184 are configured to be brought together with the interior faces 186 abutting one another to trap the plunger rod 40 within the member 180.

The member 180 is sized to extend along a length of the plunger rod 40 so that the end wall 196 provides the plunger stop 102 of the stop assembly 100 to restrict movement of the plunger rod 40. For example, the end wall 196 can be spaced from the proximal end 20 of the barrel 14 prior to use and can be configured to abut the proximal end 20 of the barrel 14 after the plunger rod 40 has traveled the distance D with respect to the barrel 14 to purge the headspace 52. With this configuration, if a user pushes on the plunger rod 40, the member 180 prevents full movement of the plunger rod 40 to prevent the product 12 from being dispensed.

The member 180 can be held in the closed configuration around the plunger rod 40 by the securing mechanism 198. In one example, the securing mechanism 198 can be an adhesive member, e.g., tape or a sticker, that secures the bodies 182, 184 together. In another example, the securing mechanism 198 can be connecting structures, such as snap-fit features, tongue-and-groove features, or the like. If desired, the bodies 182, 184 can be pivotably coupled together on one side by a hinge or other pivot and the securing mechanism 198 can couple the other side together. Alternatively, the bodies 182, 184 can be separate components and the securing mechanism 198 can couple the bodies 182, 184 together. So configured, after the headspace 52 has been purged, a user can at least partially remove the adhesive member 198 and take the member 180 off the plunger rod 40. In another example, the securing mechanism 198 can be an elastic member that extends around the member 180.

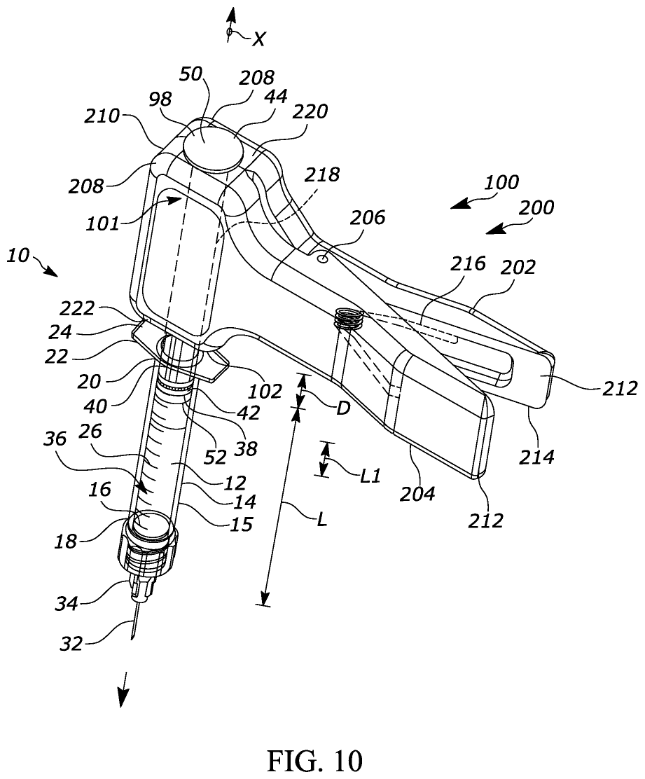

A fifth example syringe 10 is shown in FIG. 10. In this form, the stop assembly 100, including the removable portion 101 and the plunger stop 102, is provided by a clamp 200 having first and second bodies 202, 204 pivotably coupled together by a pivot 206. The first and second bodies 202, 204 each include a jaw 208 at a distal end 210 thereof and a handle 212 at a proximal end 214 thereof. The bodies 202, 204 are held with the jaws 208 in a closed configuration by a biasing device 216. So configured, a user can squeeze the handles 212 together to thereby open the jaws 208. The jaws 208 combine to define a through bore 218 extending through the clamp 200 when in the closed configuration sized to allow the plunger rod 40 to extend through the clamp 200. The bore 218 can extend entirely through the clamp 200 or can be defined by openings in the outer walls of the jaws 208. The jaws 208 are sized so that the clamp 200 is retained between the flange 48 of the engagement portion 44 and the barrel 14, with an upper surface 220 of the clamp 200 configured to abut the flange 48 of the engagement portion 44 and a lower surface 222 of the clamp 200, providing the plunger stop 102 of this form, configured to abut the proximal end 20 of the barrel 14. With this configuration, if a user pushes on the plunger rod 40, the clamp 200 restricts full movement of the plunger rod 40 to dispense the product 12. For example, the bore 218 can have at least one dimension, e.g., diameter, smaller than the flange 48 of the engagement portion 44 so that the clamp 200 is retained on the plunger rod 40 by the upper surface 220 hitting the flange 48. Additionally, the clamp 200 can be prevented from transferring to the barrel 14 by the flange 22 abutting the clamp 200 and/or the bore 218 having a dimension, e.g. diameter, smaller than the barrel 14 so that the lower surface 222 of the clamp 200 abuts the proximal end 20 of the barrel 14.

Accordingly, a user can open the jaws 208 by squeezing the handles 212 together and position the jaws 208 on either side of the plunger rod 40. The biasing device 216 closes the jaws 208 around the plunger rod 40 to attach the clamp 200 thereto. After a practitioner inspects and turns the syringe 10 upward, the practitioner can push on the plunger rod 40 until the clamp 200 restricts further movement thereof by extending between the proximal end 20 of the barrel 14 and the flange 48 of the engagement portion 44. Thereafter, the clamp 200 can be removed by opening the jaws 208. After the clamp 200 is removed, the plunger rod 40 is unrestricted for a normal administration procedure.

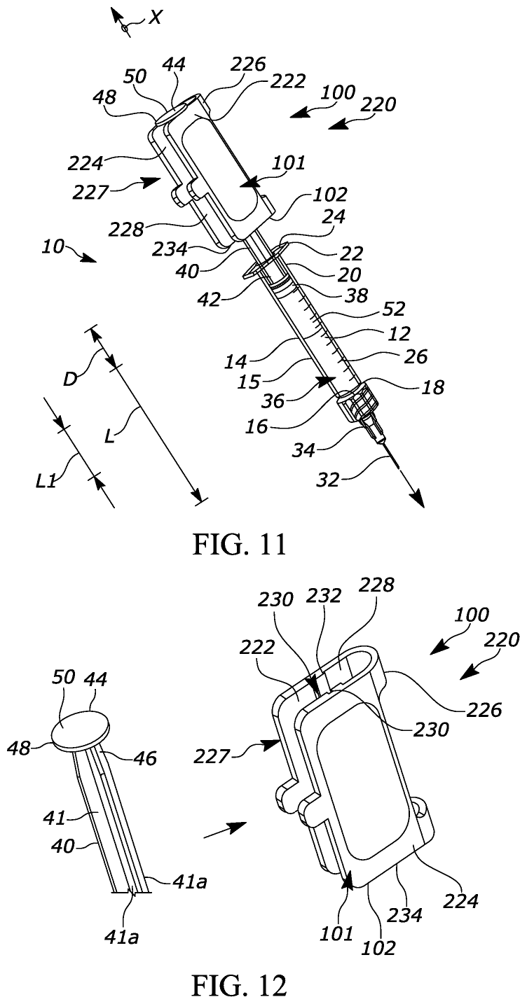

A sixth example syringe 10 is shown in FIGS. 11 and 12. In this form, the stop assembly 100, including the removable portion 101 and the plunger stop 102, is provided by a snap-fit member 220 having opposing first and second walls 222, 224 joined together by an end wall 226 and having an opposite, open end 227. The first and second walls 222, 224 are spaced apart from one another to removably receive and retain the plunger rod 40 therebetween. In the illustrated version, the plunger rod 40 has a cross-shaped horizontal cross-section with intersecting walls 41a. With this configuration, interior faces 228 of the walls 222, 224 can include a vertical channel 230 defined therein and the walls 222, 224 can be spaced apart a distance so that one of the walls 41a of the plunger rod 40 is received within and retained by the channels 230. Further, the snap-fit member 220 can be made from a material that allows the first and second walls 222, 224 to be resiliently flexed away from one another, so that the plunger rod 40 can be inserted into and removed from a storage position being retained between the walls 222, 224.

The snap-fit member 230 is sized to be retained between the flange 48 of the engagement portion 44 and the barrel 14, with an upper surface 232 of the member 230 configured to abut the flange 48 of the engagement portion 44 and a lower surface 234 of the member 230, providing the plunger stop 102 of this form, configured to abut the proximal end 20 of the barrel 14. With this configuration, if a user pushes on the plunger rod 40, the snap-fit member 230 restricts full movement of the plunger rod 40 to dispense the product 12. For example, the flange 48 of the engagement portion 44 can have a dimension larger than the space between the walls 222, 224 so that the snap-fit member 230 is retained on the plunger rod 40 by the upper surface 232 hitting the flange 48. Additionally, the snap-fit member 230 can be prevented from transferring to the barrel 14 by the flange 22 or other surface of the proximal end 20 of the barrel 14 abutting the lower surface 234.

Accordingly, a user can flex the walls 222, 224 apart from one another and position the plunger rod 40 to be received within the channels 230. The snap-fit member 220 resiliently returns to an unflexed position to thereby retain the snap-fit member 220 on the plunger rod 40. The snap-fit member 220 is sized to only allow the plunger rod 40 to be pushed into the barrel 14 the distance D that purges the headspace 52 from the syringe 10. After a practitioner inspects and turns the syringe 10 upward, the practitioner can push on the plunger rod 40 until the snap-fit member 220 restricts further movement thereof by extending between the proximal end 20 of the barrel 14 and the flange 48 of the engagement portion 44. Thereafter, the snap-fit member 220 can be removed by flexing the walls 222, 224 away from one another and removing the plunger rod 40. After the snap-fit member 220 is removed, the plunger rod 40 is unrestricted for a normal administration procedure.

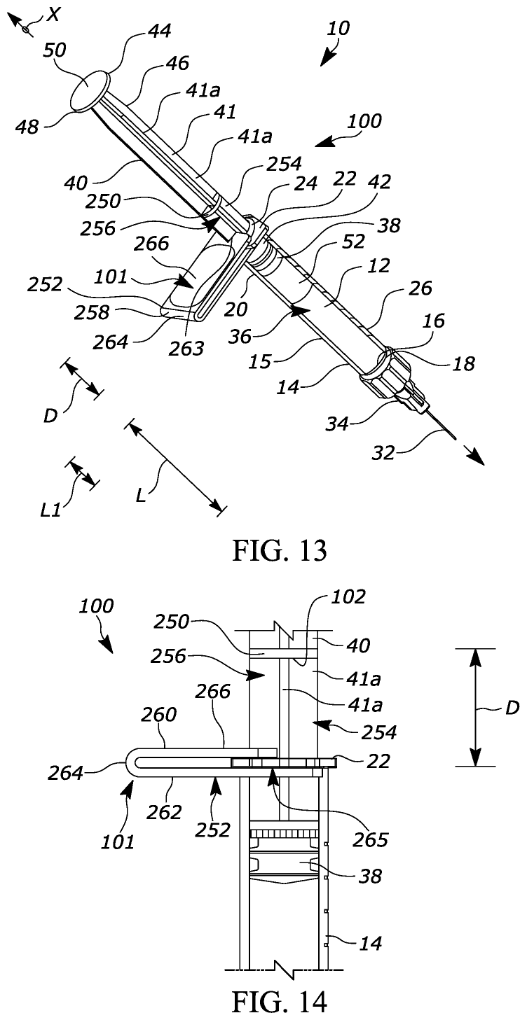

Seventh and eighth example syringes 10 are shown in FIGS. 13-16. In these forms, the stop assembly 100 includes a flange 250 of the plunger rod 40 providing the plunger stop 102 of this form that interacts with a blocking member 252 removably coupled to the barrel 14 providing the removable portion 101 of this form. In one example, the plunger rod body 41 has a x-shaped cross section formed by intersecting walls 41a. In other examples, the plunger rod body 41 can have a circular cross-section or the like having smaller dimensions than the inner diameter of the barrel 14. As is evident, in any form, the plunger rod body 41 has a cross-sectional width less than an inner diameter of the barrel 14 so that the plunger rod 40 can be inserted into the barrel 14. As shown, a least a portion 254 of the distal end 42 of the plunger rod body 41 has free space 256 between the structure of the plunger rod body 41 and a radial dimension corresponding to the inner diameter of the barrel 14. The flange 250 extends at least partially within this space 256, while being sized to fit within the barrel 14 and allow normal operation of the plunger rod 40. With this configuration, the flange 250 travels through a different path than the distal end portion 254 as the plunger rod 40 is pushed into the barrel 14. The blocking member 252 is coupled to proximal end 20 of the barrel 14 to extend at least partially into the path of the flange 250 to thereby prevent further movement of the plunger rod 40. In these forms, the flange 250 provides the plunger stop 102 of the stop assembly 100 that is spaced from an upper surface 257 of the blocking member 252, which is associated with the proximal end 20 of the barrel 14, the distance D. After the headspace 52 has been purged by driving the flange 250 into contact with the blocking member 252, the user can remove the blocking member 252 from the barrel 14, which leaves the plunger rod 40 unrestricted for a normal administration procedure.

In the seventh example syringe 10 shown in FIGS. 13 and 14, the blocking member 252 is a clip 258 having spaced first and second walls 260, 262 connected by an end wall 264 with an open, opposite end 265. The first and second walls 260, 262 are spaced apart a distance sized to receive the flange 22 of the barrel proximal end 20 therebetween. In one form, the distance between the walls 260, 262 can be sized so that the clip 258 is frictionally retained on the flange 22. Alternatively, a snap-fit, tongue-and-groove, or similar complementary structures can be utilized. So configured, the clip 258 can be removably mounted to the flange 22 to thereby couple the clip 258 to the barrel 14. In the illustrated form, the first, upper wall 260, includes a distal slot 263 to receive a portion of a wall 41a of the plunger rod 40 therein and the second, lower wall 262 is configured to extend around the barrel 14 adjacent to the flange 22. As discussed above, when the clip 258 is mounted to the flange 22, a portion of an upper surface 266 of the clip 258 is placed within the path of the flange 250 to thereby restrict movement of the plunger rod 40 after the plunger rod 40 has been driven the distance D sufficient to purge the headspace 52 in the barrel 14. After the headspace 52 has been purged, a user can simply slide the clip 258 off the flange 44.

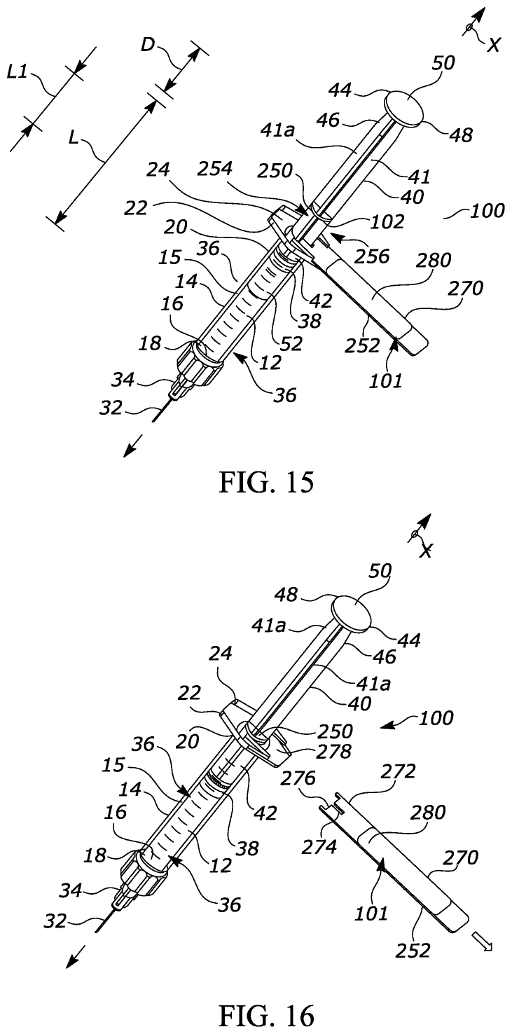

In the eighth example syringe 10 shown in FIGS. 15 and 16, the blocking member 252 is a tab 270, which can be planar as shown. In one form, the tab 270 can have an elongate configuration to provide an easy gripping portion for a user. A distal end 272 of the tab 270 has a configuration complementary to a portion of the cross-section of the plunger rod 40. For example, with the x-shaped cross-section shown, the distal end 272 includes a slot 274 to receive a portion of a protruding wall of the plunger rod 40 and lateral spaces 276 to receive the transverse wall therein, such that the tab 270 can be slid into engagement with the plunger rod 40. Of course, the distal end 272 could alternatively include a concave edge or other configuration to be complementary to the plunger rod 40. As shown, the flange 22 can include a channel 278 formed therein to receive and align the tab 270 with the plunger rod 40. In one version, the channel 278 can be sized to frictionally receive the tab 270 to hold the tab 270 in engagement with the plunger rod 40. Alternatively, or in addition thereto, the slot 274 and/or lateral spaces 276 can be sized to frictionally engage the plunger rod 40.

With this configuration, when the tab 270 is engaged to the plunger rod 40, an upper surface 280 of the tab 270 is placed within the path of the flange 250 to thereby restrict movement of the plunger rod 40 after the plunger rod 40 has been driven the distance D sufficient to purge the headspace 52 in the barrel 14. After the headspace 52 has been purged, a user can simply pull the tab 270 away from the plunger rod 40 and off the flange 22.

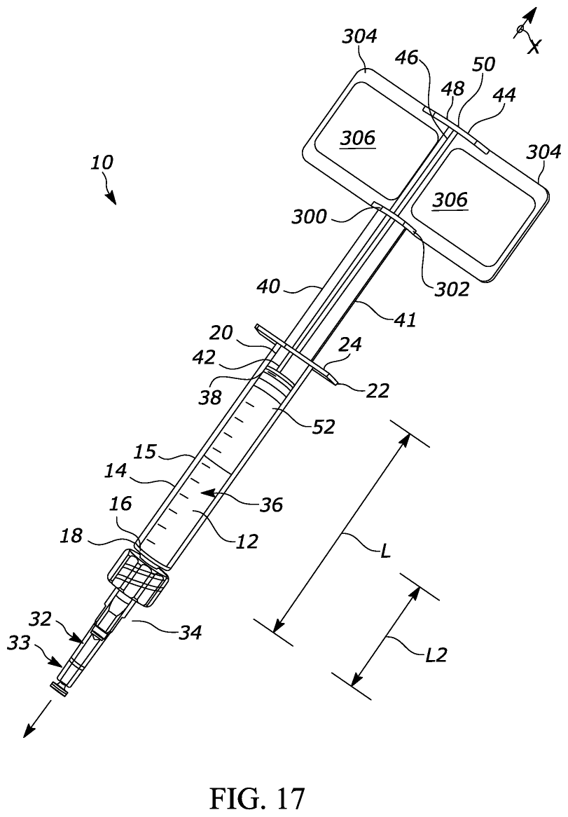

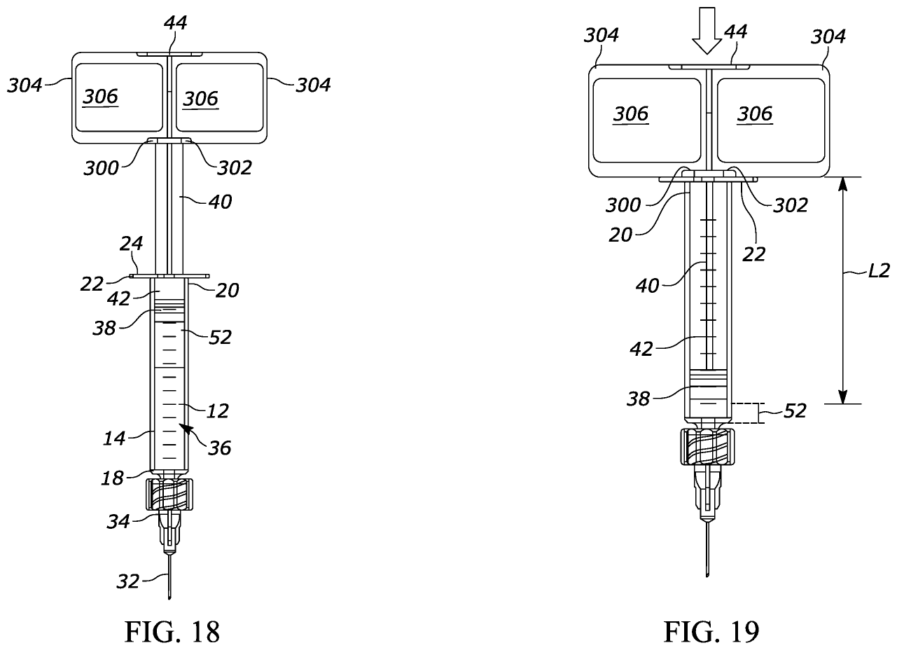

In second examples, shown in FIGS. 17-21, the syringe 10 is prepared for use with the following steps. In a first step, if applicable, the practitioner removes the needle shield 33 to expose distal end of the needle 32. In a second step, the practitioner orients the syringe 10 a generally vertical orientation, e.g., within 45 degrees of vertical and preferably within 20 degrees of vertical, with the engagement portion 44 disposed above the barrel 14 and the needle 32 pointed downward and observes the headspace 52 migrate towards the proximal end 20 of the barrel 14. As shown, each of the syringes 10 of the embodiments shown in FIGS. 17-21 include a stop 300 that restricts a distance that the plunger rod 40 can be pushed into the barrel 14. For example, the stop 300 can extend outwardly from the plunger rod body 41 a sufficient distance in a generally perpendicular direction with respect to a longitudinal axis of the plunger rod 40 to engage the end surface 24 of the barrel proximal end 20 during a dispensing operation. In this regard, the stop 300 can have at least one radial dimension greater than a corresponding radius of the opening of the barrel proximal end 20. In a third step, the practitioner can depress the plunger rod 40 to drive the stopper 38 through the barrel 14. Advantageously, the plunger rod 40 moves until the stop 300 abuts the end surface 24 of the barrel 14, stopping movement of the stopper 38 within the barrel 14. As shown in FIG. 19, the plunger rod 40 and barrel 14 are adapted so that this position of the plunger rod 14 and stopper 38 leaves the headspace 52 within the barrel 14 while dispensing a predetermined dose of the product 12.

So configured, the stop 300 prevents further displacement of the plunger rod 40 and, thus, the stopper 38, through the barrel 14 so that the dispensing operation dispenses a predetermined volume. Stated another way, by configuring the plunger rod 40 to have a predetermined length between the distal end 42 and the stop 54 that corresponds to a desired portion of the barrel 14, the syringe 10 can be preconfigured to dispense a specific volume of fluid. Advantageously, this can be utilized to avoid the problems associated with headspace within conventional pre-filled syringes by limiting a dispensing operation to a volume that prevents the headspace 52 from being expelled.

The predetermined length of the plunger rod 40 between the distal end 42 and the stop 300, combined with a length of the stopper 38, corresponds to a portion L2 of the length L of the barrel 14. This length L2 is shown in FIG. 19 with the plunger rod 40 fully inserted into the barrel 14 and the stop 300 abutting the end surface 24. Accordingly, during preparation of the syringe 10, the barrel 14 can be filled to a desired amount or dosage of the product 12 and the stopper 38 can be positioned within the barrel 14 so that the headspace 52 has a length, and corresponding volume, generally equal to a remaining portion L2 of the length L of the barrel 14.

Of course, the fluid of the product 12 may interact with the barrel 14 to have a meniscus rather than a flat top surface. In order to ensure that none of the headspace 52 is dispensed, the length L2 can have a value equal to or greater than a length of the therapeutic product 12 at its raised periphery adjacent to the barrel 14.

In a first example syringe 10 of this form shown in FIGS. 17-19, the stop 300 is provided by a flange 302 extending away from the plunger rod body 41 in a direction generally perpendicular, e.g., between about 1 degree and 5 degrees, with respect to a longitudinal axis of the plunger rod 40. In one example, the flange 302 can extend radially outwardly away from an entire perimeter of the plunger rod body 41, such as with a circular or polygonal configuration. Alternatively, the flange 302 can extend outwardly away from portions of the perimeter.

Additionally, as shown, the plunger rod 40 can include one or more wall portions 304 that extend longitudinally between the stop flange 302 and the flange 48 of the engagement portion 44 to provide main faces 306 visible along the longitudinal axis of the syringe 10. If desired, the wall portions 304 can extend laterally outwardly away from the plunger rod body 41 a greater distance than the flanges 302, 48, such that the main surfaces 306 provide extended surface areas for the provision of instructions, drug identification information, or other information thereon for easy viewing by a user. For example, the information can inform a user as to the proper operation and use of the syringe 10. The information can be printed directly on the main surfaces 306 and/or can be applied thereto, such as with a sticker or the like.

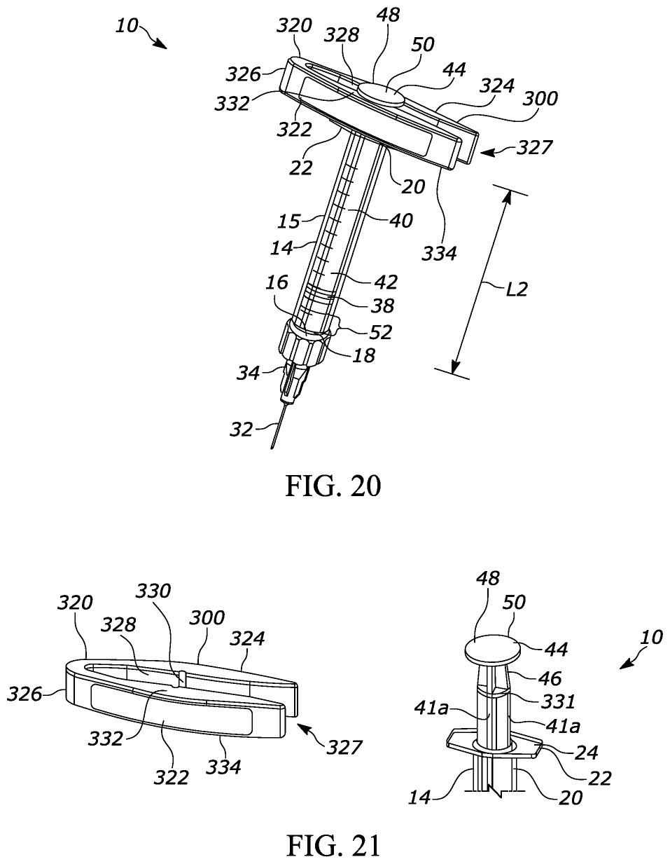

In a second example syringe 10 of this form shown in FIGS. 20 and 21, the stop 300 is provided by a snap-fit member 320 having opposing first and second walls 322, 324 joined together by an end wall 326 with an open, opposite end 327. The first and second walls 322, 324 are spaced apart from one another a distance to removably receive and retain the plunger rod 40 therebetween. In the illustrated version, the plunger rod 40 has a cross-shaped horizontal cross-section with intersecting walls 41a. With this configuration, interior faces 328 of the walls 322, 324 can include a vertical channel 330 defined therein and the walls 322, 324 can be spaced apart a distance so that one of the walls 41a of the plunger rod 40 is received within and retained by the channels 330. Further, the snap-fit member 320 can be made from a material that allows the first and second walls 322, 324 to be resiliently flexed away from one another, so that the plunger rod 40 can be inserted into and removed from a storage position being retained between the walls 322, 324. If desired, the plunger rod 40 can include a flange 331 having a perimeter sized to fit within the barrel 14 that is spaced a distance from the engagement portion 44 substantially equal to a height of the snap-fit member 320 to retain the snap-fit member 320 on a discrete portion of the plunger rod 40.

The snap-fit member 320 is sized to be retained between the flange 48 of the engagement portion 44 and the barrel 14, with an upper surface 332 of the member 320 configured to abut the flange 48 of the engagement portion 44 and a lower surface 334 of the member 320 configured to abut the proximal end 20 of the barrel 14. With this configuration, if a user pushes on the plunger rod 40, the snap-fit member 320 restricts full movement of the plunger rod 40, only allowing a practitioner to dispense the desired dose of the product 12. For example, the flange 48 of the engagement portion 44 can have a dimension larger than the space between the walls 322, 324 so that the snap-fit member 320 is retained on the plunger rod 40 by the upper surface 332 hitting the flange 48. Additionally, the snap-fit member 320 can be prevented from transferring to the barrel 14 by the flange 22 or other surface of the proximal end 20 of the barrel 14 abutting the lower surface 334.

Accordingly, a user can flex the walls 322, 324 apart from one another and position the plunger rod 40 to be received within the channels 330. The snap-fit member 320 resiliently returns to an unflexed position to thereby retain the snap-fit member 320 on the plunger rod 40. The snap-fit member 320 has a height sized to only allow the plunger rod 40 to be pushed into the barrel 14 the distance L2 that administers the products 12 and leaves the headspace 52 in the syringe 10.

The disclosure provided herein can be utilized with any desired dosage and syringe size, while ensuring a delivery amount for accurate dosages. This is suitable for pre-filled syringes for patient self-injection or for practitioner-administered injection. The components of the syringe 10, such as the barrel 14 and plunger rod 40, can be made of any suitable material, such as plastic or glass. In one example, the syringes 10 described herein can provide a desired accuracy of fluid therapeutic product injection of +/−10 microliters. In a 1 mL syringe, the movement of the plunger can be controlled to +/−0.3 mm to achieve this desired accuracy.

It will be appreciated that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments. The same reference numbers may be used to describe like or similar parts. Further, while several examples have been disclosed herein, any features from any examples may be combined with or replaced by other features from other examples. Moreover, while several examples have been disclosed herein, changes may be made to the disclosed examples within departing from the scope of the claims.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing di merization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, 90-threonine], Darbepoetin alfa, novel erythropoiesis stimulating protein (NESP); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4β7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Solids™ (eculizumab); pexelizumab (anti-05 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP Ilb/Ilia receptor monoclonal antibody); Actemra® (anti-IL6 Receptor mAb); Avastin® (bevacizumab), HuMax-CD4 (zanolimumab); Mvasi™ (bevacizumab-awwb); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 145c7-CHO (anti-IL15 antibody, see U.S. Pat. No. 7,153,507); Tysabri® (natalizumab, anti-α4integrin mAb); Valortim® (MDX-1303, anti-B. anthracis protective antigen mAb); ABthrax™ Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Ra mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MY0-029); anti-GM-CSF Receptor mAb (CAM-3001); anti-HepC mAb (HuMax HepC); anti-IFNα mAb (MEDI-545, MDX-198); anti-IGF1R mAb; anti-IGF-1R mAb (HuMax-Inflam); anti-IL12 mAb (ABT-874); anti-IL12/1L23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCG8 mAb (MDX-1307); anti-mesothelin dsFv-PE38 conjugate (CAT-5001); anti-PD1mAb (MDX-1106 (ONO-4538)); anti-PDGFRa antibody (IMC-3G3); anti-TGFβ mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity™ (romosozumab-aqqg), another product containing romosozumab for treatment of postmenopausal osteoporosis and/or fracture healing and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. In some embodiments, the drug delivery device may contain or be used with Aimovig® (erenumab-aooe), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BITE®) antibodies such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with Avsola™ (infliximab-axxq), anti-TNF a monoclonal antibody, biosimilar to Remicade® (infliximab) (Janssen Biotech, Inc.) or another product containing infliximab for the treatment of autoimmune diseases. In some embodiments, the drug delivery device may contain or be used with Kyprolis® (carfilzomib), (2S)-N-((S)-1-((S)-4-methyl-1-((R)-2-methyloxiran-2-yl)-1-oxopentan-2-ylcarbamoyl)-2-phenylethyl)-2-((S)-2-(2-morpholinoacetamido)-4-phenylbutanamido)-4-methylpentanamide, or another product containing carfilzomib for the treatment of multiple myeloma. In some embodiments, the drug delivery device may contain or be used with Otezla® (apremilast), N-[2-[(1S)-1-(3-ethoxy-4-methoxyphenyl)-2-(methylsulfonyl)ethyl]-2,3-dihydro-1,3-dioxo-1H-isoindo1-4-yl]acetamide, or another product containing apremilast for the treatment of various inflammatory diseases. In some embodiments, the drug delivery device may contain or be used with Parsabiv™ (etelcalcetide HCl, KAI-4169) or another product containing etelcalcetide HCl for the treatment of secondary hyperparathyroidism (sHPT) such as in patients with chronic kidney disease (KD) on hemodialysis. In some embodiments, the drug delivery device may contain or be used with ABP 798 (rituximab), a biosimilar candidate to Rituxan®/MabThera™, or another product containing an anti-CD20 monoclonal antibody. In some embodiments, the drug delivery device may contain or be used with a VEGF antagonist such as a non-antibody VEGF antagonist and/or a VEGF-Trap such as aflibercept (Ig domain 2 from VEGFR1 and Ig domain 3 from VEGFR2, fused to Fc domain of IgG1). In some embodiments, the drug delivery device may contain or be used with ABP 959 (eculizumab), a biosimilar candidate to Soliris®, or another product containing a monoclonal antibody that specifically binds to the complement protein C5. In some embodiments, the drug delivery device may contain or be used with Rozibafusp alfa (formerly AMG 570) is a novel bispecific antibody-peptide conjugate that simultaneously blocks ICOSL and BAFF activity. In some embodiments, the drug delivery device may contain or be used with Omecamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Sotorasib (formerly known as AMG 510), a KRASG12c small molecule inhibitor, or another product containing a KRASG12c small molecule inhibitor. In some embodiments, the drug delivery device may contain or be used with Tezepelumab, a human monoclonal antibody that inhibits the action of thymic stromal lymphopoietin (TSLP), or another product containing a human monoclonal antibody that inhibits the action of TSLP. In some embodiments, the drug delivery device may contain or be used with AMG 714, a human monoclonal antibody that binds to Interleukin-15 (IL-15) or another product containing a human monoclonal antibody that binds to Interleukin-15 (IL-15). In some embodiments, the drug delivery device may contain or be used with AMG 890, a small interfering RNA (siRNA) that lowers lipoprotein(a), also known as Lp(a), or another product containing a small interfering RNA (siRNA) that lowers lipoprotein(a). In some embodiments, the drug delivery device may contain or be used with ABP 654 (human IgG1 kappa antibody), a biosimilar candidate to Stelara®, or another product that contains human IgG1 kappa antibody and/or binds to the p40 subunit of human cytokines interleukin (IL)-12 and IL-23. In some embodiments, the drug delivery device may contain or be used with Amjevita™ or Amgevita™ (formerly ABP 501) (mab anti-TNF human IgG1), a biosimilar candidate to Humira®, or another product that contains human mab anti-TNF human IgG1. In some embodiments, the drug delivery device may contain or be used with AMG 160, or another product that contains a half-life extended (HLE) anti-prostate-specific membrane antigen (PSMA)×anti-CD3 BiTE® (bispecific T cell engager) construct. In some embodiments, the drug delivery device may contain or be used with AMG 119, or another product containing a delta-like ligand 3 (DLL3) CART (chimeric antigen receptor T cell) cellular therapy. In some embodiments, the drug delivery device may contain or be used with AMG 119, or another product containing a delta-like ligand 3 (DLL3) CART (chimeric antigen receptor T cell) cellular therapy. In some embodiments, the drug delivery device may contain or be used with AMG 133, or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 199 or another product containing a half-life extended (HLE) bispecific T cell engager construct (BITE®). In some embodiments, the drug delivery device may contain or be used with AMG 256 or another product containing an anti-PD-1×IL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 330 or another product containing an anti-CD33×anti-CD3 BiTE® (bispecific T cell engager) construct. In some embodiments, the drug delivery device may contain or be used with AMG 404 or another product containing a human anti-programmed cell death-1(PD-1) monoclonal antibody being investigated as a treatment for patients with solid tumors. In some embodiments, the drug delivery device may contain or be used with AMG 427 or another product containing a half-life extended (HLE) anti-fms-like tyrosine kinase 3 (FLT3)×anti-CD3 BiTE® (bispecific T cell engager) construct. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAP×4-1BB-targeting DARPin® biologic under investigation as a treatment for solid tumors. In some embodiments, the drug delivery device may contain or be used with AMG 509 or another product containing a bivalent T-cell engager and is designed using XmAb® 2+1 technology. In some embodiments, the drug delivery device may contain or be used with AMG 562 or another product containing a half-life extended (HLE) CD19×CD3 BiTE® (bispecific T cell engager) construct. In some embodiments, the drug delivery device may contain or be used with Efavaleukin alfa (formerly AMG 592) or another product containing an IL-2 mutein Fc fusion protein. In some embodiments, the drug delivery device may contain or be used with AMG 596 or another product containing a CD3×epidermal growth factor receptor vlIl (EGFRvIll) BiTE® (bispecific T cell engager) molecule. In some embodiments, the drug delivery device may contain or be used with AMG 673 or another product containing a half-life extended (HLE) anti-CD33×anti-CD3 BiTE® (bispecific T cell engager) construct. In some embodiments, the drug delivery device may contain or be used with AMG 701 or another product containing a half-life extended (HLE) anti-B-cell maturation antigen (BCMA)×anti-CD3 BiTE® (bispecific T cell engager) construct. In some embodiments, the drug delivery device may contain or be used with AMG 757 or another product containing a half-life extended (HLE) anti-delta-like ligand 3 (DLL3)×anti-CD3 BiTE® (bispecific T cell engager) construct. In some embodiments, the drug delivery device may contain or be used with AMG 910 or another product containing a half-life extended (HLE) epithelial cell tight junction protein claudin 18.2×CD3 BiTE® (bispecific T cell engager) construct.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A syringe comprising:
a barrel having an interior, a dispensing opening at a distal end, and an open proximal end;
a stopper disposed within the interior of the barrel;
a plunger rod having a first end configured to be at least selectively operably coupled with the stopper and a second end extending through the open proximal end of the barrel, the second end including an outwardly extending flange; and
a stop assembly including a plunger stop coupled to the plunger rod at a location between the flange and the open proximal end of the barrel and a removable portion coupled to the plunger rod or barrel, the plunger stop being configured and dimensioned to engage a stop surface adjacent to the open proximal end of the barrel to selectively limit displacement of the plunger and stopper along the interior of the barrel.

2. The syringe of claim 1, wherein the plunger stop and the removable portion of the stop assembly comprises first and second bodies pivotable with respect to one another to capture the plunger rod therebetween.

3. The syringe of claim 2, wherein the plunger stop and the removable portion of the stop assembly comprises a clip including a hinge pivotably coupling first sides of the first and second bodies and a securing mechanism releasably connecting second sides of the first and second bodies.

4. The syringe of claim 2, wherein the plunger stop and the removable portion of the stop assembly comprises a clamp including a biasing mechanism, and the first and second bodies comprise jaws at first ends thereof and handles at second ends thereof, the biasing mechanism biasing the jaws of the first and second bodies together to capture the plunger rod therebetween.

5. The syringe of claim 1, wherein the plunger stop and the removable portion of the stop assembly comprises a tab coupled to the plunger rod along a longitudinal length thereof by a breakaway feature.

6. The syringe of claim 1, wherein the plunger stop and the removable portion of the stop assembly comprises:
a body defining a cavity with an opening through an end wall thereof, the cavity configured to receive the plunger rod therein to restrict longitudinal movement of the plunger rod with respect to the body; and
a securing mechanism coupled to the body to hold the plunger rod within the cavity.

7. The syringe of claim 6, wherein the securing mechanism comprises an adhesive member extending over cavity.

8. The syringe of claim 6, wherein the body comprises first and second clamshell components having inner faces configured to be brought together to define the cavity therebetween, and the securing mechanism holds the first and second clamshell components in a closed configuration with the plunger rod trapped within the cavity.

9. The syringe of claim 1, wherein the plunger stop and the removable portion of the stop assembly comprises a snap-fit member comprising first and second walls spaced from one another and connected by an end wall, the first and second walls defining channels on interior faces thereof and being configured to be flexed apart to dispose the plunger rod therebetween and returned towards an unflexed position to retain portions of the plunger rod within the channels.

10. The syringe of claim 1, wherein the proximal end of the barrel comprises an outwardly projecting flange, and the removable portion of the stop assembly comprises a blocking member configured to removably engage the flange of the barrel; and wherein the plunger stop of the stop assembly comprises a stop flange extending outwardly from the plunger rod adjacent to the first end thereof, the stop flange having a perimeter sized to fit within the interior of the barrel and the blocking member having a portion disposed within a travel path of the stop flange to restrict longitudinal movement of the plunger rod.

11. The syringe of claim 10, wherein the blocking member comprises a clip having first and second walls spaced from one another and connected by an end wall, the clip configured to engage the flange of the barrel between the first and second walls.

12. The syringe of claim 10, wherein the blocking member comprises a tab having a distal end with a configuration complementary to a portion of a horizontal cross-section of the plunger rod, and the flange of the barrel comprises a channel sized to receive the tab therein, the channel configured to guide the tab into engagement with the plunger rod.

13. The syringe of claim 1, wherein the stop assembly comprises a wall portion having a face with information displayed thereon regarding the operation of the stop assembly.

14. A method of dispensing headspace from a syringe containing a liquid therapeutic product, the method comprising:
providing a syringe comprising: a barrel having an interior, a dispensing opening at a distal end, and an open proximal end; a stopper disposed within the interior of the barrel; a plunger rod having a first end configured to be at least selectively operably coupled with the stopper and a second end extending through the open proximal end of the barrel, wherein the second end includes an outwardly extending flange; a fluid therapeutic product and a headspace disposed in the interior of the barrel between the stopper and the dispensing opening; and a stop assembly including a plunger stop coupled to the plunger rod and a removable portion coupled to the plunger rod or barrel;
driving the plunger rod toward the open proximal end of the barrel of the syringe to drive the stopper along the interior of the barrel toward the dispensing opening to dispense the headspace through the dispensing opening;
abutting the plunger stop against a stop surface adjacent to the open proximal end of the barrel, thereby stopping movement of the stopper and preventing the fluid therapeutic product from being expelled; and
removing the removable portion of the stop assembly from the plunger rod or barrel.

15. The method of claim 14, wherein the plunger stop and the removable portion of the stop assembly comprises first and second bodies having the plunger rod captured therebetween, and removing the removable portion of the stop assembly from the plunger rod or barrel comprises pivoting first and second bodies of the stop with respect to one another.

16. The method of claim 14, wherein removing the removable portion of the stop assembly from the plunger rod or barrel comprises breaking a tab off of the plunger rod.

17. The method of claim 14, wherein the plunger stop and the removable portion of the stop assembly comprises: a body defining a cavity with an opening through an end wall thereof, the plunger rod received within the cavity, and a securing mechanism coupled to the body to hold the plunger rod within the cavity, and removing the removable portion of the stop assembly from the plunger rod or barrel comprises manipulating the body and securing mechanism to free the plunger rod from the cavity.

18. The method of claim 14, wherein removing the removable portion of the stop assembly from the plunger rod or barrel comprises flexing first and second walls of the stop assembly away from one another to release the plunger rod from being captured therebetween.

19. The method of claim 14, wherein the proximal end of the barrel comprises an outwardly projecting flange, the plunger stop comprises a stop flange extending outwardly from the plunger rod, wherein the stop flange has a perimeter sized to fit within the interior of the barrel, and removing the removable portion of the stop assembly from the plunger rod or barrel comprises uncoupling a blocking member having a portion disposed within a travel path of the stop flange to restrict longitudinal movement of the plunger rod from the flange of the barrel.

20. A syringe comprising:
a barrel having an interior, a dispensing opening at a distal end, and an open proximal end having an end surface;
a stopper disposed within the interior of the barrel;
a plunger rod having a first end configured to be at least selectively operably coupled with the stopper and a second end extending through the open proximal end of the barrel; and either (a) or (b):
(a) an outwardly projecting stop adjacent the second end of the plunger rod; an engagement portion at the second end of the plunger rod spaced from the stop; and at least one wall portion extending longitudinally between the stop and the engagement portion and laterally outward from the plunger rod, the at least one wall portion being integrally formed with the plunger rod and having a face with information displayed thereon regarding the operation of the stop, or
(b) an engagement portion at the second end of the plunger rod; a flange adjacent to the second end of the plunger rod and spaced from the engagement portion; and a snap-fit member releasably coupled to the plunger rod, the snap-fit member comprising first and second walls spaced from one another and connected by an end wall, the first and second walls being configured to be flexed apart to dispose the plunger rod therebetween and returned towards an unflexed position to retain the snap-fit member on the plunger rod between the engagement portion and the flange.

21-23. (canceled)
